(19) United States
(12) Patent Application Publication
(10) Pub. No.: US 2025/0353810 A1
(43) Pub. Date: Nov. 20, 2025

(54) PROCESS FOR THE PREPARATION OF TROFINETIDE

(71) Applicant: ZYDUS LIFESCIENCES LIMITED, Gandhinagar (IN)

(72) Inventors: Kumar Kamlesh Singh, Gandhinagar (IN); Anand Vijaykumar Mantri, Gandhinagar (IN); Mrinal Kalita, Gandhinagar (IN); Prashant Kailash Chandole, Gandhinagar (IN)

(21) Appl. No.: 19/204,039

(22) Filed: May 9, 2025

(30) Foreign Application Priority Data
May 15, 2024 (IN) 202421038202

(51) Int. Cl. C07C 231/10; C07D 207/24

(52) U.S. Cl. CPC C07C 231/10; C07D 207/24

(57) ABSTRACT

Present invention relates to Trofinetide of Formula I having a purity of 99.0% or more and one or more of each of compound of Formula II, compound of Formula A, compound of Formula B, compound of Formula C, compound of Formula D, compound of Formula E, or compound of Formula F,

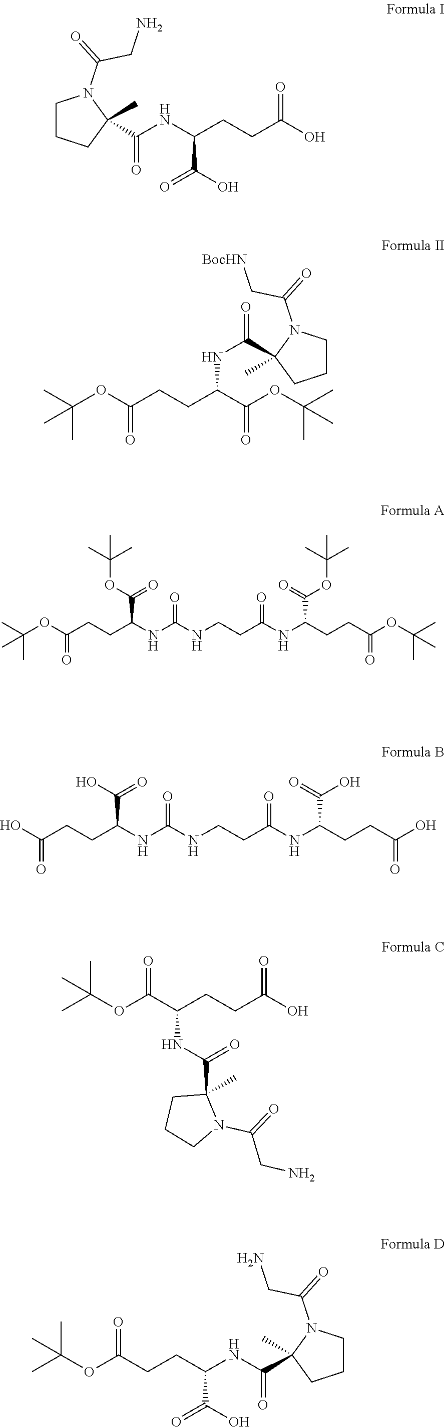

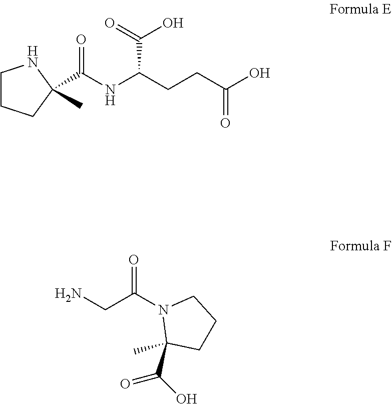 [SMILES: C[C@@]1(C(=O)N[C@@H](CCC(=O)O)C(=O)O)CCCN1 ; C[C@@]1(C(=O)O)CCCN1C(=O)CN]

which when present, is in a detectable amount of about 0.15% or less, as measured by area percentage of HPLC. In particular, the present invention relates to novel intermediate compound of Formula II, process for the preparation of novel intermediate and use of novel intermediate compound in the preparation of trofinetide. Present invention further relates to use of trofinetide in the preparation of composition comprising trofinetide.

### FIELD OF THE INVENTION

Present invention relates to Trofinetide having a purity of 99.0% or more and one or more of each of compound of Formula II, compound of Formula A, compound of Formula B, compound of Formula C, compound of Formula D, compound of Formula E, or compound of Formula F in a detectable amount of about 0.15% or less, as measured by area percentage of HPL C. In particular, the present invention relates to novel intermediate compound of Formula II, process for the preparation of novel intermediate and use of novel intermediate compound in the preparation of trofinetide. Present invention further relates to use of trofinetide in the preparation of composition comprising trofinetide.

### BACKGROUND OF THE INVENTION

Background description includes information that may be useful in understanding the present invention. It is not an admission that any of the information provided herein is prior art or relevant to the presently claimed invention, or that any publication specifically or implicitly referenced is prior art.

Trofinetide is an analog of glycine-proline-glutamate (GPE) and is IUPAC name is (2S)-2-{[(2S)-1-(2-aminoacetyl)-2-methylpyrrolidine-2-carbonyl]amino}pentanedioic acid, having chemical structure of compound of Formula I:

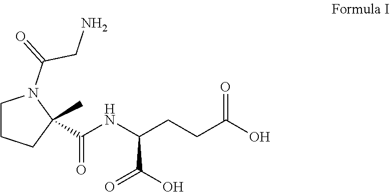 [SMILES: C[C@@]1(C(=O)N[C@@H](CCC(=O)O)C(=O)O)CCCN1C(=O)CN]

International (PCT) Publication No. 2002/094856 A 2 discloses GPE analogs and peptidomimetics including trofinetide, method of preparation of trofinetide and method of using trofinetide to treat diseases.

International (PCT) Publication No. 2007/106555 A 2 discloses oral formulation of trofinetide and use of trofinetide in treating neurodegenerative conditions.

International (PCT) Publication No. 2021/026066 A 1 provides a composition of trofinetide, wherein trofinetide is prepared using different process.

Currently, there are very limited disclosures available for the synthesis of trofinetide and intermediates thereof. Therefore, there is a need to develop cost effective and novel processes for preparation of trofinetide, which results into high overall yield and purity having high throughput and easily scalable for large scale productions.

The present invention relates to a process for the preparation of trofinetide. The present invention also provides novel intermediate compound and process for the preparation of novel intermediate compound. Further, the present invention provides use of novel intermediate compound in the preparation of trofinetide, composition comprising trofinetide and use of composition comprising trofinetide in the treatment of Rett syndrome.

### SUMMARY OF THE INVENTION

In one general aspect, present invention relates to trofinetide having a purity of 99.0% or more, as measured by area percentage of HPLC. In one general aspect, present invention relates to a process for the preparation of trofinetide. In one general aspect, present invention relates to a novel intermediate compound of Formula II, process for the preparation of novel intermediate compound of Formula II and use of novel intermediate compound of Formula II in the preparation of trofinetide. In another general aspect, present invention further relates to use of trofinetide in the preparation of composition comprising trofinetide and their use in treatment of Rett syndrome.

In one general aspect, present invention relates to trofinetide having a purity of 99.0% or more, as measured by area percentage of HPLC.

In one general aspect, present invention relates to trofinetide having a purity of 99.0% or more, and one or more of each of compound of Formula II, compound of Formula A, compound of Formula B, compound of Formula C, compound of Formula D, compound of Formula E, or compound of Formula F,

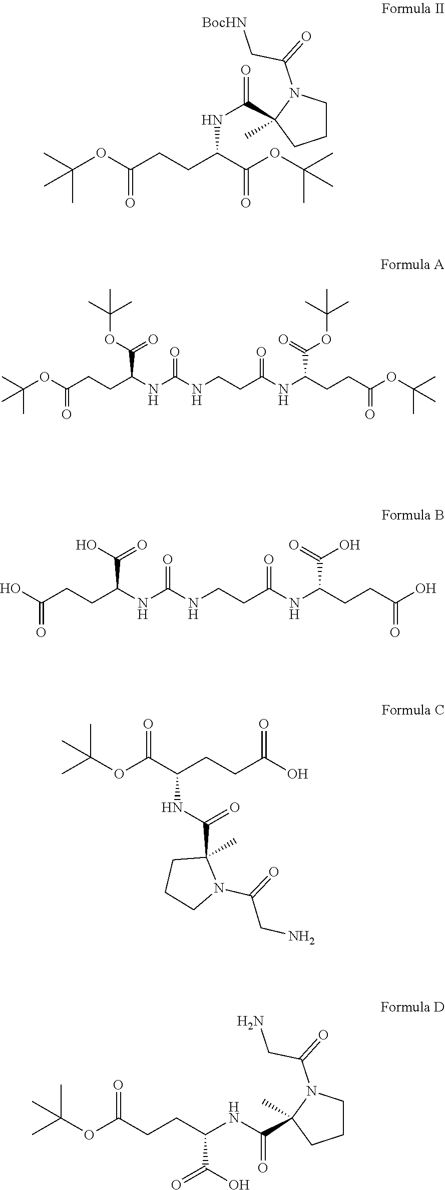

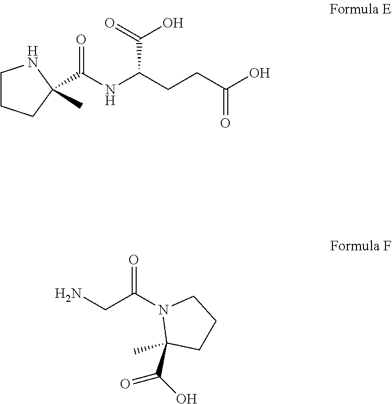 [SMILES: C[C@@]1(C(=O)N[C@@H](CCC(=O)O)C(=O)O)CCCN1 ; C[C@@]1(C(=O)O)CCCN1C(=O)CN]

which when present, is in a detectable amount of about 0.15% or less, as measured by area percentage of HPLC.

In one general aspect, there is provided a process for the preparation trofinetide, or salts, or hydrates thereof. The process comprising
(a) reacting a compound of Formula VII,

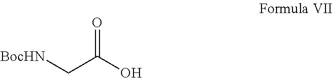 [SMILES: CC(C)(C)OC(=O)NCC(=O)O]

with N-Hydroxysuccinimide in the presence of one or more reagents to obtain a compound of Formula VI;

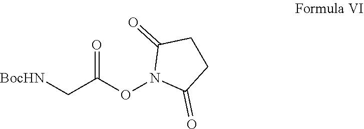 [SMILES: CC(C)(C)OC(=O)NCC(=O)ON1C(=O)CCC1=O]

(b) reacting the compound of Formula VI with a compound of Formula V or salts thereof,

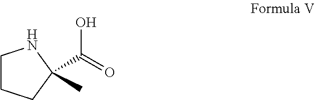

in the presence of one or more bases to obtain a compound of Formula IV;

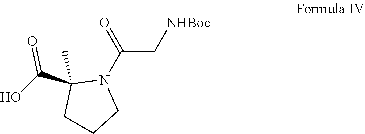 [SMILES: CC(C)(C)OC(=O)NCC(=O)N1CCC[C@@]1(C)C(=O)O]

(c) reacting the compound of Formula IV with a compound of Formula III or salts

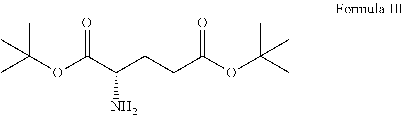 [SMILES: CC(C)(C)OC(=O)CC[C@H](N)C(=O)OC(C)(C)C]

in the presence of one or more reagents to obtain a compound of Formula Il;

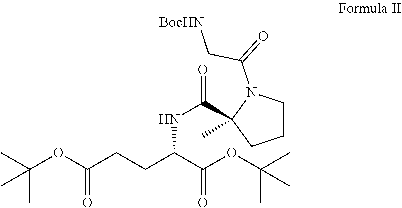 [SMILES: CC(C)(C)OC(=O)CC[C@H](NC(=O)[C@]1(C)CCCN1C(=O)CNC(=O)OC(C)(C)C)C(=O)OC(C)(C)C]

(d) deprotecting the compound of Formula II with a reagent to obtain trofinetide compound and
(e) optionally, converting trofinetide obtained in step (d) to salts or hydrates thereof.

In another general aspect, there is provided a novel compound of Formula II,

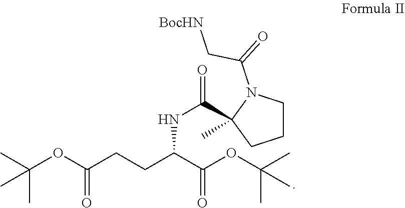 [SMILES: CC(C)(C)OC(=O)CC[C@H](NC(=O)[C@]1(C)CCCN1C(=O)CNC(=O)OC(C)(C)C)C(=O)OC(C)(C)C]

In another general aspect, there is provided compound of Formula II is used in the preparation of trofinetide,

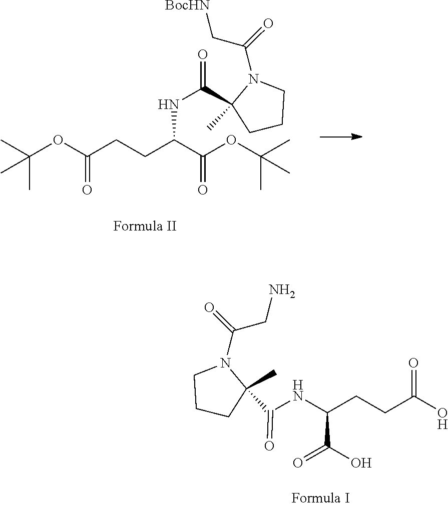

or salts, or hydrates thereof.

In another general aspect there is provided a process for the preparation of novel intermediate compound of Formula II. The process comprising:
(a) reacting a compound of Formula VII,

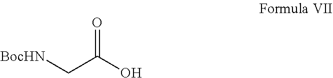 [SMILES: CC(C)(C)OC(=O)NCC(=O)O]

with N-Hydroxysuccinimide in the presence of one or more reagents to obtain a compound of Formula VI;

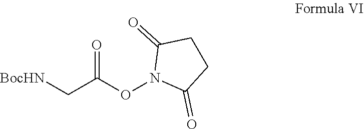 [SMILES: CC(C)(C)OC(=O)NCC(=O)ON1C(=O)CCC1=O]

(b) reacting the compound of Formula VI with a compound of Formula V or salts thereof,

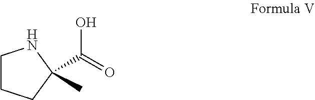

in the presence of one or more bases to obtain a compound of Formula IV;

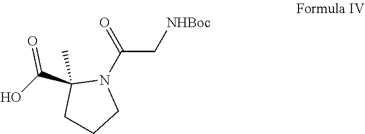 [SMILES: CC(C)(C)OC(=O)NCC(=O)N1CCC[C@@]1(C)C(=O)O]

(c) reacting the compound of Formula IV with a compound of Formula III or salts thereof,

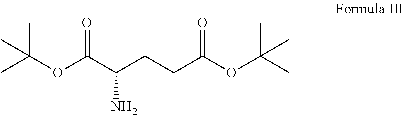 [SMILES: CC(C)(C)OC(=O)CC[C@H](N)C(=O)OC(C)(C)C]

in the presence of one or more reagents to obtain a compound of Formula II,

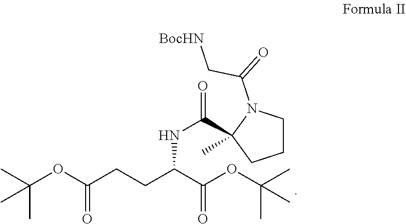 [SMILES: CC(C)(C)OC(=O)CC[C@H](NC(=O)[C@]1(C)CCCN1C(=O)CNC(=O)OC(C)(C)C)C(=O)OC(C)(C)C]

In another general aspect, present invention provides trofinetide or salts or hydrates thereof having a purity of about 98.0% or more by area percentage of HPLC. Particularly, trofinetide having a purity of about 99.0% or more, more particularly, a purity of about 99.5% or more, further more particularly, a purity of about 99.8% or more, most particularly, a purity of about 99.9% or more, by area percentage of HPLC.

In another general aspect, present invention provides trofinetide or salts or hydrates thereof having a chiral purity of about 98.0% or more, by area percentage of HPLC. Particularly, trofinetide having a chiral purity of about 99.0% or more, more particularly, a chiral purity of about 99.5% or more, further more particularly, a chiral purity of about 99.8% or more, most particularly, a chiral purity of about 99.9% or more, by area percentage of HPLC.

In another general aspect, the present invention provides a composition comprising trofinetide or pharmaceutically acceptable salts or hydrates thereof with pharmaceutically acceptable carrier.

In another general aspect, the present invention provides a composition comprising trofinetide or pharmaceutically acceptable salt thereof having purity of about 99.0% or more as determined by area percentage of HPLC.

In another general aspect, the present invention provides a composition comprising trofinetide or pharmaceutically acceptable salt thereof having chiral purity of about 99.0% or more as determined by area percentage of HPLC.

In another general aspect, the present invention provides a use of composition comprising trofinetide in the treatment of Rett syndrome.

### DEFINITIONS

The term “solution” as used herein, unless described otherwise, does not necessarily mean only a clear solution or one wherein the solute is completely soluble in the solvent; all the intermediate phases of mixture of components, starting from a state wherein the solute has started getting dissolved in the solvent to a state wherein the solute has completely dissolved in the solvent, are included within the expression ‘solution’.

As used herein, the term “area percentage of HPLC” refers to the area percentage of a peak in HPLC chromatogram. In general, the area percentage calculation procedure reports the area of each peak in the chromatogram as a percentage of the total area of all peaks.

The terms ‘obtaining’, ‘isolating’, and ‘purifying’ are generally interchangeable and include, but not limited to, decantation, filtration, extraction, evaporation, crystallization, recrystallization and chromatographic operations.

Optionally, the solution, prior to any solids formation and/or solvent removal, can be filtered to remove any undissolved solids, particulate matter or solid impurities. Any filtration system and filtration techniques known in the art can be used for the purpose.

Terms such as “about”, and “generally”, are to be construed as modifying a term or value to which they are attached such that the term or the value is not absolute. This includes, at very least, the degree of expected experimental error, technique error and instrument error for a given technique used to measure a value.

The term “salt” or “salts” includes salts with an inorganic acid e.g. hydrochloric acid, hydroiodic acid, phosphoric acid, phosphonic acid, sulfuric acid, hydrobromic acid, or with an organic acid e.g. formic acid, acetic acid, citric acid, malic acid, maleic acid, tartaric acid, succinic acid, hemisuccinic acid, salicylic acid, trifluoroacetic acid, trichloroacetic acid, oxalic acid, benzoic acid, p-toluene sulfonic acid or methanesulfonic acid.

The term “pharmaceutically acceptable” as used herein is intended to mean that it is useful in preparing a pharmaceutical composition that is generally non-toxic and is not biologically undesirable, and includes that which is acceptable for veterinary use and/or human pharmaceutical use.

The term “composition” used herein means a physical mixture of two or more components.

The term “pharmaceutical composition” is intended to encompass a drug product including the active ingredient(s), pharmaceutically acceptable excipients that make up the carrier, as well as any product, which results, directly or indirectly, from combination, complexation or aggregation of any two or more of the ingredients. Accordingly, the pharmaceutical compositions encompass any composition made by admixing the active ingredient, active ingredient dispersion or composite, additional active ingredient(s), and pharmaceutically acceptable excipients.

The terms used throughout the description is defined herein below.
“HOBt” refers 1-hydroxy-benzotriazole
“HOAt” refers 1-hydroxy-azabenzotriazole
“HATU” refers Hexafluorophosphate Azabenzotriazole Tetramethyl Uronium
“HBTU” refers Hexafluorophosphate Benzotriazole Tetramethyl Uronium
“TBTU” refers 2-(1H-benzotriazole-1-yl)-1,1,3,3-tetramethylaminium tetrafluoroborate
“TPTU” refers O-(1,2-Dihydro-2-oxo-1-pyridyl)-N,N,N′-N′-tetramethyluronium tetrafluoroborate
“TOTU” refers O-((ethoxycarbonyl)cyanomethyleneamino)-N,N,N′,N′-tetramethyluronium tetrafluoroborate
“EDC” refers 1-Ethyl-3-(3-dimethylaminopropyl)carbodiimide
“EDC.HCl” refers 1-Ethyl-3-(3-dimethylaminopropyl)carbodiimide hydrochloride
“DCC” refers N,N′-dicyclohexylcarbodiimide
“DIC” refers N,N′-diisopropylcarbodiimide
“MTBE” refers methyl tert-butyl ether
“TFA” refers trifluoroacetic acid
“HCl” refers hydrochloric acid
“TEA” refers triethyl amine
“DIPEA” refers N,N-diisopropylethylamine
“DMAP” refers 4-dimethylaminopyridine
“NaHCO₃” refers sodium bicarbonate
“Na₂SO₄” refers Sodium sulfate
“TIPS” refers triisopropyl silane
“Boc” refers di-tert-butyl decarbonate
“NMM” refers N-methylmorpholine
“HPLC” refers High Performance Liquid Chromatography
“PyBOP” refers benzotriazol-1-yloxytripyrrolidinophosphonium hexafluorophosphate
“T 3P” refers Propanephosphonic acid anhydride
“BOP” refers benzotriazol-1-yloxytris (dimethylamino) phosphonium hexafluorophosphate
“CDI” refers 1′-Carbonyldiimidazole
“COMU” refers 1-Cyano-2-ethoxy-2-oxoethylidenaminooxy) dimethylamino-morpholino-carbenium hexafluorophosphate
“DMI” refers 1,3-Dimethyl-2-imidazolidinone
“gm” refers gram
“Gly” refers Glycine
“Trofinetide” refers glycyl-L-2-methylprolyl-L-glutamic acid of compound of Formula I or H-Gly-MePro-Glu-OH.
“Boc-Gly-OH” refers N-(tert-Butoxycarbonyl)glycine
“Boc-Gly-OSu” refers Boc-glycine N-hydroxysuccinimide ester
“Boc-Gly-MePro-OH” refers 1-((tert-butoxycarbonyl)glycyl)-2-methylpyrrolidine-2-carboxylic acid
“H-Glu(OtBu)-OtBu” refers di-tert-butyl L-glutamate hydrochloride
“Boc-Gly-MePro-Glu(OtBu)-OtBu” refers di-tert-butyl ((S)-1-((tert-butoxycarbonyl)glycyl)-2-methylpyrrolidine-2-carbonyl)-L-glutamate

### DETAILED DESCRIPTION OF THE INVENTION

The aforementioned general and further aspects of the invention are achieved by the detailed description of the invention provided herein after.

In one general aspect, present invention relates to trofinetide having a purity of 99.5% or more, as measured by area percentage of HPLC.

In one general aspect, present invention relates to trofinetide having a purity of 99.0% or more, and one or more of each of compound of Formula II, compound of Formula A, compound of Formula B, compound of Formula C, compound of Formula D, compound of Formula E, or compound of Formula F,

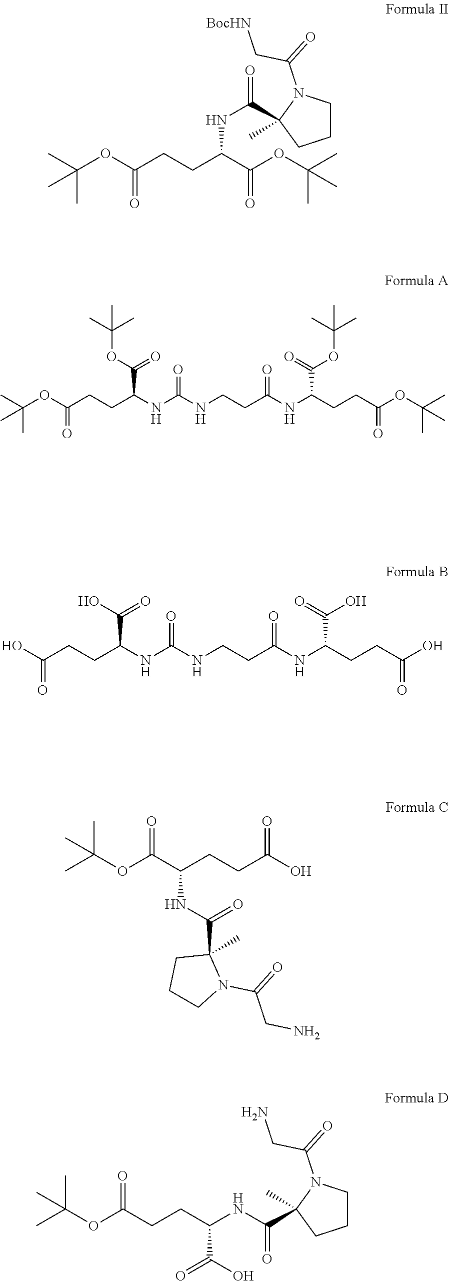

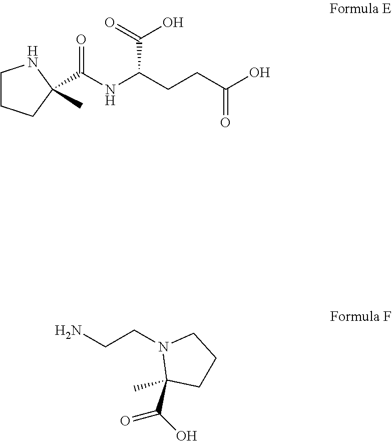 [SMILES: C[C@@]1(C(=O)N[C@@H](CCC(=O)O)C(=O)O)CCCN1 ; C[C@@]1(C(=O)O)CCCN1CCN]

which when present, is in a detectable amount of about 0.15% or less, as measured by area percentage of HPLC.

In one general aspect, trofinetide relates to crystalline trofinetide or amorphous trofinetide. In one general aspect, trofinetide is in the form of salts, hydrates, or solvates thereof.

In one general aspect, there is provided a process for the preparation trofinetide.

The process comprising
(a) reacting a compound of Formula VII,

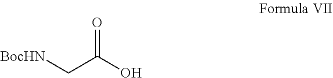 [SMILES: CC(C)(C)OC(=O)NCC(=O)O]

with N-Hydroxysuccinimide in the presence of one or more reagents to obtain a compound of Formula VI;

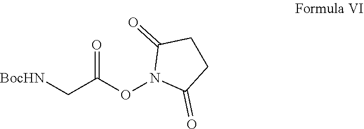 [SMILES: CC(C)(C)OC(=O)NCC(=O)ON1C(=O)CCC1=O]

(b) reacting the compound of Formula VI with a compound of Formula V or salts thereof,

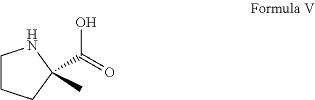

in the presence of one or more bases to obtain a compound of Formula IV;

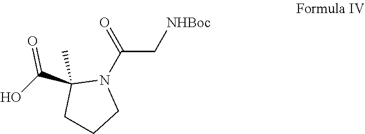 [SMILES: CC(C)(C)OC(=O)NCC(=O)N1CCC[C@@]1(C)C(=O)O]

(c) reacting the compound of Formula IV with a compound of Formula III or salts

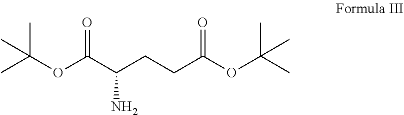 [SMILES: CC(C)(C)OC(=O)CC[C@H](N)C(=O)OC(C)(C)C]

in the presence of one or more reagents to obtain a compound of Formula II;

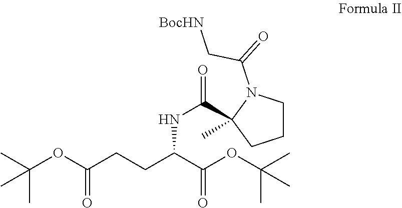 [SMILES: CC(C)(C)OC(=O)CC[C@H](NC(=O)[C@]1(C)CCCN1C(=O)CNC(=O)OC(C)(C)C)C(=O)OC(C)(C)C]

(d) deprotecting the compound of Formula II with a reagent to obtain a trofinetide compound and
(e) optionally, converting trofinetide obtained in step (d) to salts or hydrates thereof.

In another general aspect, there is provided a novel compound of Formula II,

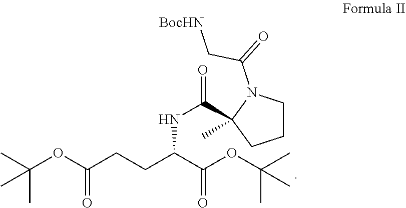 [SMILES: CC(C)(C)OC(=O)CC[C@H](NC(=O)[C@]1(C)CCCN1C(=O)CNC(=O)OC(C)(C)C)C(=O)OC(C)(C)C]

In another general aspect, there is provided compound of Formula II is used in the preparation of trofinetide,

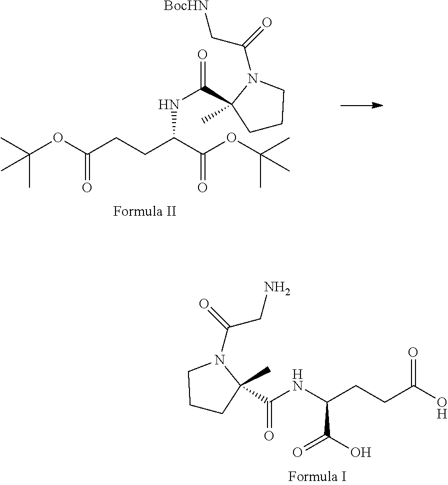 [SMILES: CC(C)(C)OC(=O)CC[C@H](NC(=O)[C@]1(C)CCCN1C(=O)CNC(=O)OC(C)(C)C)C(=O)OC(C)(C)C ; C[C@@]1(C(=O)N[C@@H](CCC(=O)O)C(=O)O)CCCN1C(=O)CN]

or salts, or hydrates thereof.

In another general aspect there is provided a process for the preparation of novel intermediate compound of Formula II. The process comprising:
(a) reacting a compound of Formula VII,

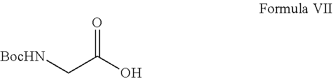 [SMILES: CC(C)(C)OC(=O)NCC(=O)O]

with N-Hydroxysuccinimide in the presence of one or more reagents to obtain a compound of Formula VI;

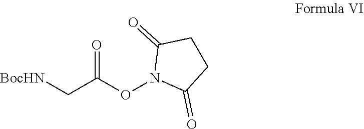 [SMILES: CC(C)(C)OC(=O)NCC(=O)ON1C(=O)CCC1=O]

(b) reacting the compound of Formula VI with a compound of Formula V or salts thereof,

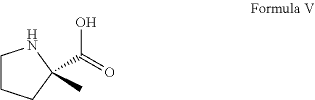

in the presence of one or more bases to obtain a compound of Formula IV;

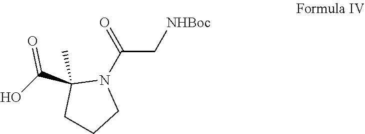 [SMILES: CC(C)(C)OC(=O)NCC(=O)N1CCC[C@@]1(C)C(=O)O]

(c) reacting the compound of Formula IV with a compound of Formula III or salts thereof,

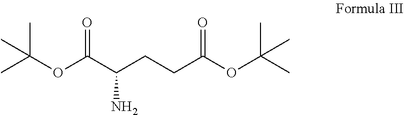 [SMILES: CC(C)(C)OC(=O)CC[C@H](N)C(=O)OC(C)(C)C]

in the presence of one or more reagents to obtain a compound of Formula II,

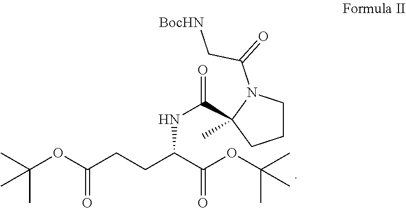 [SMILES: CC(C)(C)OC(=O)CC[C@H](NC(=O)[C@]1(C)CCCN1C(=O)CNC(=O)OC(C)(C)C)C(=O)OC(C)(C)C]

In another general aspect, present invention provides trofinetide having compound of compound of Formula II, Formula A, compound of Formula B, compound of Formula C, compound of Formula D, compound of Formula E, or compound of Formula F below 0.5% or less by area percentage of HPLC. Particularly, trofinetide having compound of Formula II, compound of Formula A, compound of Formula B, compound of Formula C, compound of Formula D, compound of Formula E, or compound of Formula F below 0.2% or less by area percentage of HPLC, preferably trofinetide having compound of Formula II, compound of Formula A, compound of Formula B, compound of Formula C, compound of Formula D, compound of Formula E, or compound of Formula F below 0.05% or less by area percentage of HPLC and more preferably trofinetide having compound of Formula II, compound of Formula A, compound of Formula B, compound of Formula C, compound of Formula D, compound of Formula E, or compound of Formula F in no detectable level,

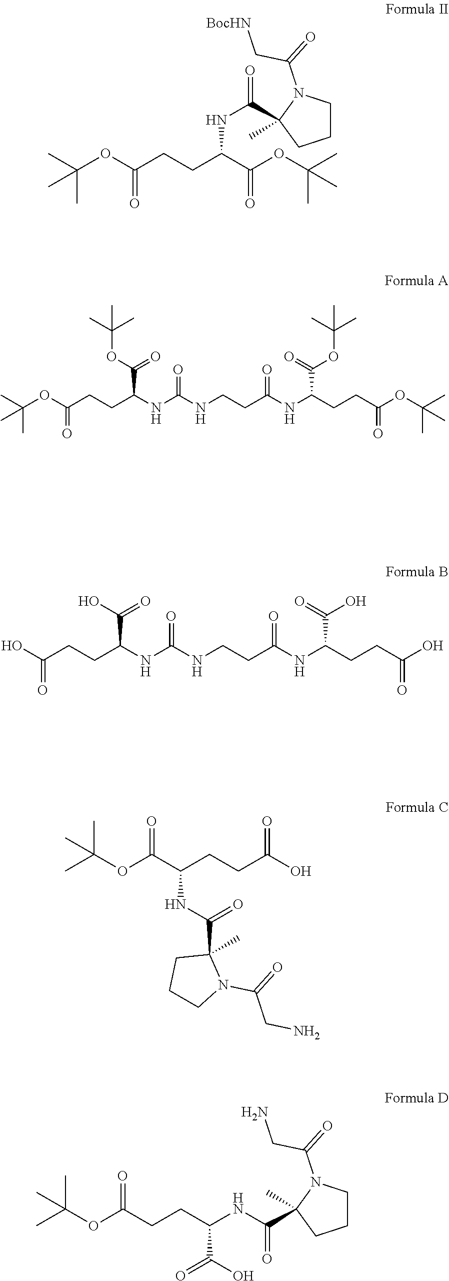

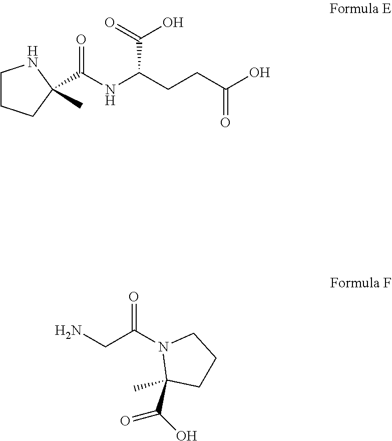 [SMILES: C[C@@]1(C(=O)N[C@@H](CCC(=O)O)C(=O)O)CCCN1 ; C[C@@]1(C(=O)O)CCCN1C(=O)CN]

as measured by area percentage of HPLC.

In general, the reactions at step (a), (b), (c), (d) and (e) may be performed in the presence of one or more solvent selected from acetonitrile, tetrahydrofuran, 2-methyltetrahydrofuran, xylene, methanol, ethanol, propanol, isopropyl alcohol, dichloromethane, toluene, ethyl acetate, dimethylacetamide, n-heptane, n-hexane, dioxane, di-n-butyl ether, dimethylformamide, N-Methyl-2-pyrrolidone, MTBE, dimethyl ether, water, or mixtures thereof.

In general, the reaction at (a) may be performed in presence of one or more reagents selected from HOBt, HOAt, Oxyma, DMAP, HATU, HBTU, TBTU, TPTU, TOTU, EDC, EDC.HCl, DCC, DIC, PyBOP, T3P, BOP, CDI, Methyl chloroformate, COMU, or mixtures thereof.

In general, the base at step (b) may be performed in presence of one or more bases selected from one or more of TEA, DIPEA, NMM, DMI, pyridine, N-methylpiperidine, NaHCO₃, sodium carbonate, or mixtures thereof

In general, the reaction at step (c) may be performed in presence of one or more reagent selected from one or more of HOBt, HOAt, Oxyma, DMAP, HATU, HBTU, TBTU, TPTU, TOTU, EDC, EDC.HCl, DCC, DIC, PyBOP, T3P, BOP, CDI, Methyl chloroformate, COMU, DMAP, TEA, DIPEA, NMM, pyridine, N-methylpiperidine, or mixtures thereof.

In general, the reaction at step (d) may be performed in presence of one or more reagent selected from one or more of TFA, HCl, trimethylsilyl iodide, or mixtures thereof.

The process of this aspect may be performed with isolation or without isolation of the intermediate compounds or salts or hydrates thereof, which is further converted to trofinetide.

Optionally, the compound of the present invention can be purified further. The compounds of present invention can be in isolated and/or purified form, but such is not required.

The trofinetide may be isolated by precipitation, evaporation, spray drying, or other conventional techniques known in the art.

In another general aspect, the present invention provides the analytical method for the determination of compound of Formula A, compound of Formula B, compound of Formula C, compound of Formula D, compound of Formula E and compound of Formula F in trofintide.

### Method for Compound of Formula I

Instrument Name: Waters HPLC e2992 system equipped with UV/PDA detector Software: Empower 3.0

### Description of Analytical Method

Chromatographic conditions:
Equipment: Waters HPLC e2992 system equipped with UV/PDA detector
Column: Inertsustain AQ C18 (250×4.6) 5μ or equivalent
Detector: UV-VIS or PDA
Wavelength: 210 nm
Flow Rate: 0.5 ml/min
Column temp.: 40° C.
Sampler cooler temp.: 5° C.
Injection Volume: 20 mL
Run time: 130 minutes

### Gradient Programme

| Time (minutes) | % Mobile phase B |
|---|---|
| 0 | 0 |
| 5 | 0 |
| 10 | 5 |
| 35 | 15 |
| 40 | 35 |
| 100 | 85 |
| 110 | 85 |
| 111 | 0 |
| 130 | 0 |

### Diluent

Preparation of Mobile phase-A: Prepare degassed mixture of 0.05% Perchloric acid (70%) in water.

Preparation of Mobile phase-B: Prepare degassed mixture of Water, Acetonitrile and Perchloric acid (70%) in the ratio of 20:80:0.05 (% v/v/v).

In another general aspect, present invention provides trofinetide or salts or hydrates thereof having a purity of about 98.0% or more by area percentage of HPLC. Particularly, trofinetide having a purity of about 99.0% or more, more particularly, a purity of about 99.5% or more, further more particularly, a purity of about 99.8% or more, most particularly, a purity of about 99.9% or more, by area percentage of HPLC.

In another general aspect, present invention provides trofinetide or salts or hydrates thereof having a chiral purity of about 98.0% or more, by area percentage of HPLC. Particularly, trofinetide having a chiral purity of about 99.0% or more, more particularly, a chiral purity of about 99.5% or more, further more particularly, a chiral purity of about 99.8% or more, most particularly, a chiral purity of about 99.9% or more, by area percentage of HPLC.

In another general aspect, the present invention provides a composition comprising trofinetide or pharmaceutically acceptable salts or hydrates thereof having a purity of 99.0% or more, as measured by area percentage of HPLC. Particularly, composition comprising trofinetide or pharmaceutically acceptable salts or hydrates having a purity of about 99.5% or more, more particularly, purity of about 99.8% or more and most particularly, a purity of about 99.9% or more by area percentage of HPLC.

In another general aspect, the present invention provides a composition comprising trofinetide or pharmaceutically acceptable salts or hydrates thereof having a purity of 99% or more and one or more of each of compound of Formula II, compound of Formula A, compound of Formula B, compound of Formula C, compound of Formula D, compound of Formula E, or compound of Formula F,

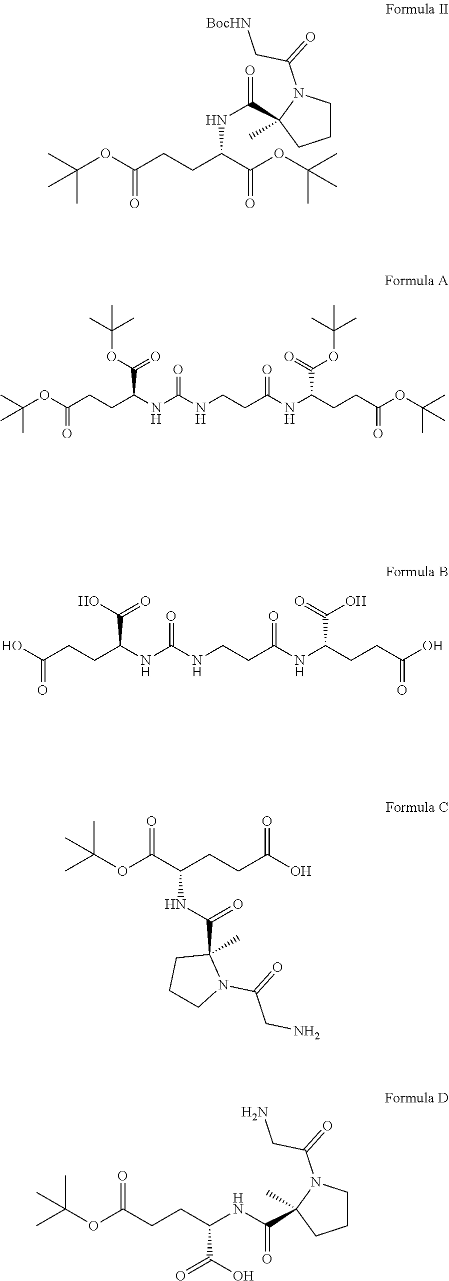

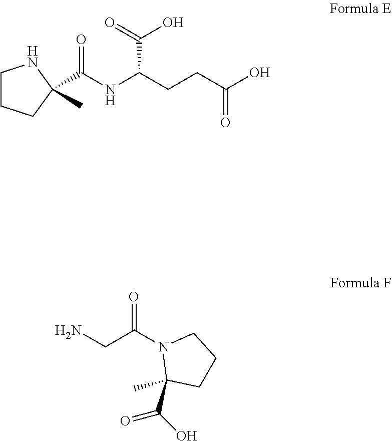 [SMILES: C[C@@]1(C(=O)N[C@@H](CCC(=O)O)C(=O)O)CCCN1 ; C[C@@]1(C(=O)O)CCCN1C(=O)CN]

which when present, is in a detectable amount of about 0.15% or less, as measured by area percentage of HPLC.

In another general aspect, the present invention provides a pharmaceutical composition comprising trofinetide or pharmaceutically acceptable salts or hydrates thereof with pharmaceutically acceptable carrier.

In another general aspect, the present invention provides a pharmaceutical composition comprising trofinetide or pharmaceutically acceptable salts or hydrates thereof having a purity of 99.0% or more, as measured by area percentage of HPLC. Particularly, pharmaceutical composition comprising trofinetide or pharmaceutically acceptable salts or hydrates having a purity of about 99.5% or more, more particularly, purity of about 99.8% or more and most particularly, purity of about 99.9% or more by area percentage of HPLC.

In another general aspect, the present invention provides a pharmaceutical composition comprising trofinetide or pharmaceutically acceptable salt thereof having chiral purity of about 99.5% or more as determined by area percentage of HPLC with pharmaceutically acceptable carrier.

In another general aspect, the present invention provides a pharmaceutical composition comprising trofineti de or pharmaceutically acceptable salts or hydrates thereof having a purity of 99.5% or more and one or more of each of compound of Formula II, compound of Formula A, compound of Formula B, compound of Formula C, compound of Formula D, compound of Formula E, or compound of Formula F,

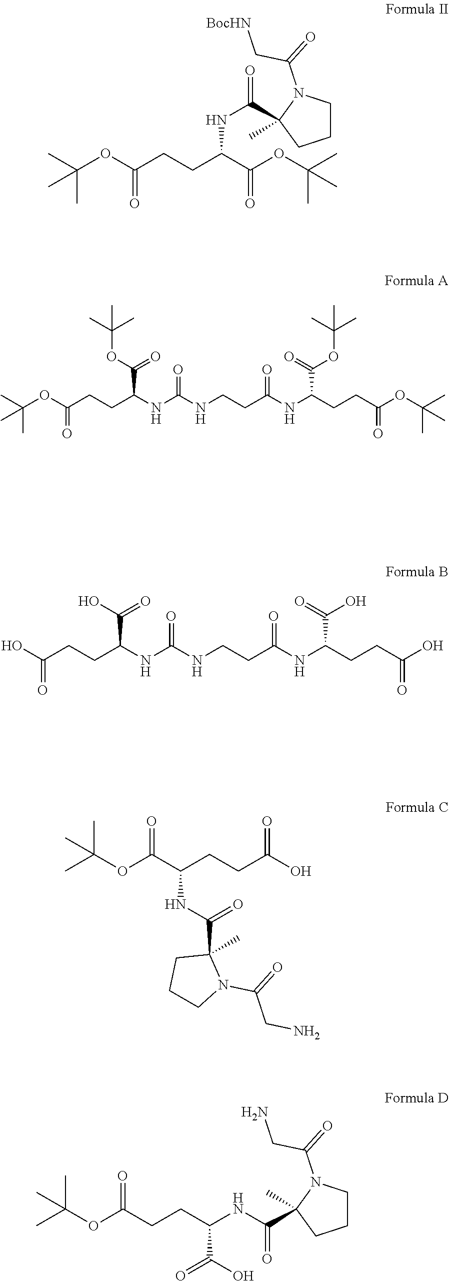

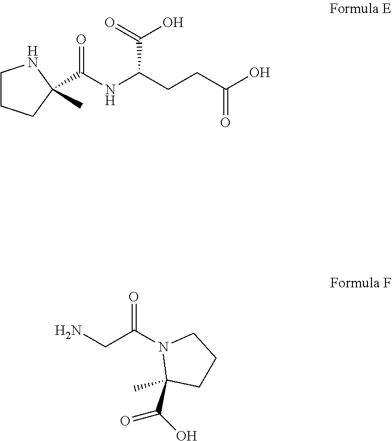 [SMILES: C[C@@]1(C(=O)N[C@@H](CCC(=O)O)C(=O)O)CCCN1 ; C[C@@]1(C(=O)O)CCCN1C(=O)CN]

which when present, is in a detectable amount of about 0.15% or less, as measured by area percentage of HPLC with pharmaceutically acceptable carrier.

In another general aspect, the present invention provides an use of composition comprising trofinetide in the treatment of Rett syndrome.

The present invention is further illustrated by following reaction examples which are provided merely to be representative of the invention and do not limit the scope of it.

### EXAMPLES

### Preparation of Trofinetide (H-GLY-MEPRO-GLU-OH)

### Example 1: Preparation of BOC-GLY-OSU

To a 4 neck round bottom flask, Boc-Gly-OH (20 gm) and N-Hydroxysuccinimide (15.74 gm) was dissolved in isopropanol (200 mL) at room temperature. Further, EDC.HCl (26.22 gm) was added to the reaction mixture and allowed to stir for 1 hour at room temperature. Upon completion of reaction, reaction mixture was cooled to get below 15° C. The final product was filtered, washed with isopropyl alcohol (300 mL) and dried under vacuum to obtain title compound. Yield: 28.23 gm (91.06%).

### Example 2: Preparation of BOC-GLY-MEPRO-OH

To a 4 neck round bottom flask, 2-Methyl-L-Proline Hydrochloride (30.3 gm) was added into acetonitrile (1000 mL). Further DIPEA (63.7 mL) and Boc-Gly-OSu (50 gm) was added subsequently into the reaction mixture. The suspension was heated up to 45° C. and allowed to stir for 4 hours. After completion of reaction, reaction mixture was cooled to get room temperature. A cetonitrile was evaporated under vacuum from the reaction mixture. Ethyl acetate (500 mL) was added into the residue and reaction mass was twice washed with 5% aq. NaHCO₃ (500 mL). The aqueous layer was acidified with 12N aq. HCl at room temperature (pH˜3) and extracted with ethyl acetate (1000 mL). The ethyl acetate layer was washed with water (1000 mL) and brine 500 mL followed by drying over anhydrous Na₂SO₄. The reaction was filtered and solvent was removed in vacuo. Cyclohexane (500 mL) was added to the suspension and stirred for 30 min at 10-15° C. Reaction mass was filtered and washed with cyclohexane (500 mL) and dried under vacuum to obtain title compound. Purity by HPLC: 99.65%; Yield: 36 gm (68.5%).

### Example 3: Preparation of BOC-GLY-MEPRO-GLU (OTBU)-OTBU

To a 4 neck round bottom flask, Boc-Gly-MePro-OH (30 gm) was added into dichloromethane (300 mL). EDC.HCl (29.9 gm) and DMAP (3.8 gm) was added into reaction mass and stirred for 10 min. H-Glu(OtBu)-OtBu hydrochloride (33.8 gm) followed by DIPEA (54.3 mL) was added into the reaction mass and reaction mixture was stirred for 4 hour. After completion of reaction, reaction mixture was washed with 4N aq. HCl (600mL) followed by 5% aq. NaHCO₃ (600mL) and water (300 mL). The organic layer was dried over anhydrous Na₂SO₄, filtered and the solvent was evaporated under vacuum. n-Heptane (300 mL) was charged into suspension and stirred for 30 min at room temperature. Reaction mixture was filtered, washed with n-Heptane (300 mL) and dried under vacuum to obtain title compound. Purity by HPLC: 99.45%; Yield: 37.2 gm (67.3%).

### Example 4: Preparation of Trofinetide (H-GLY-MEPRO-GLU-OH)

### Example 4(A)

To a 4 neck round bottom flask, Boc-Gly-MePro-Glu (OtBu)-OtBu (15 gm) was charged into the deprotection solution of TFA:TIPS:Water (8:1:1, 150mL) and stirred for 4 hour at room temperature. Reaction mass was evaporated and acetonitrile (300 mL) was charged into the residue to dissolve the sticky mass. pH of the reaction mass was adjusted to 6.5-7.5 by using trimethylamine. Reaction mass was then stirred for 30 minutes, filtered, washed with acetonitrile (150 mL) and dried under vacuum to obtained title compound trofinetide. Purity by HPLC: 98.59%; Yield: 8.9 gm (96%).

### Example 4(B)

To a 4 neck round bottom flask, deprotection solution of TFA:Water:TIPS:DTT (90:8:1:1, 100 mL) was charged. Deprotection solution was cooled to get −5 to 5° C. Boc-Gly-MePro-Glu (OtBu)-OtBu (20 g) was dissolved into 20 mL dichloromethane and charged into deprotection solution at −5 to 5° C. and stirred for 4 hours at room temperature. Reaction mass was charged into the chilled methyl tertiary butyl ether (600 mL). Reaction mass was then stirred for 60 minutes at 0-10° C., filtered, washed with methyl tertiary butyl ether (100) to obtain solid. The isolated solid was dissolved in to mixture of 2-M ethyltetrahydrofuran and methanol (300 mL+20 mL), pH of the reaction mass was adjusted to 5.5-6.5 by using trimethylamine. Reaction mass was then stirred for 30 minutes, filtered and washed with 2-methyltetrahydrofuran (50 mL) and dried under vacuum to obtained title compound trofinetide. Purity by HPLC: 97.57%; Yield: 9.4 gm (85%).

### Example 4(c)

To a 4 neck round bottom flask, deprotection solution of TFA:Water:TIPS:DTT (90:8:1:1, 200 mL) was charged. Charge Boc-Gly-MePro-Glu (OtBu)-OtBu (20 g) into above deprotection solution at 25 to 35° C. and stirred for 4 hours at room temperature. Reaction mass was distilled under vacuum till oily residue at 35-40° C. 2-methyltetrahydrofuran (300 mL) was charged into oily residue. pH of the reaction mass was adjusted to 7.5-8.5 by using trimethylamine. Reaction mass was then stirred for 30 minutes, filtered, washed with 2-methyltetrahydrofuran (100 mL). The isolated solid was dried under vacuum to obtained crude trofinetide.

### Example 5: Purification of Trofinetide (H-GLY-MEPRO-GLU-OH)

### Example 5(A)

To a 4 neck round bottom flask, trofinetide (3 gm) was dissolved in methanol (15 mL). Acetonitrile (45 mL) was added slowly at ambient temperature in above solution and stirred for 30 min. Reaction mass was filtered, washed with acetonitrile (30 mL) and dried under vacuum to obtain trofinetide. Yield: 2.6 gm (86.6% %), Purity: 99.07%; compound of Formula A: ND, compound of Formula B: ND, compound of Formula C: ND, compound of Formula D: ND, compound of Formula E: 0.12%, compound of Formula F: ND.

### Example 5(B)

To a 4 neck round bottom flask, trofinetide (4.4 gm) was dissolved in methanol (13.2 mL). Above solution was charged slowly into isopropyl alcohol (33 mL) at ambient temperature and stirred for 30 min. Reaction mass was filtered, washed with isopropyl alcohol (16.5 mL) and dried under vacuum to obtain trofinetide. Yield: 3.0 gm (68%), Purity: 99.11%, compound of Formula A: ND, compound of Formula B: ND, compound of Formula C: 0.18%, compound of Formula D: 0.17%, compound of Formula E: 0.17%, compound of Formula F: ND.

### Example 5(C)

To a 4 neck round bottom flask, trofinetide (50 gm) was dissolved in methanol (150 mL). Above solution was charged slowly into isopropyl alcohol (375 mL) at ambient temperature and stirred for 30 min. Reaction mass was filtered, washed with isopropyl alcohol (185 mL) and dried under vacuum to obtain trofinetide. Yield: 34 gm (68%), Purity: 99.42%, compound of Formula A: ND, compound of Formula B: ND, compound of Formula C: 0.12%, compound of Formula D: 0.12%, compound of Formula E: 0.11%, compound of Formula F: ND.

### Example 5(D)

To a 4 neck round bottom flask, trofinetide (5 gm) was dissolved in methanol (15 mL). Above solution was charged slowly into 2-methyltetrahydrofuran (50 mL) at ambient temperature and stirred for 30 min. Reaction mass was filtered and washed with 2-methyltetrahydrofuran (25 mL) and dried under vacuum to obtain trofinetide. Yield: 3.4 g (85%), Purity: 99.70%, compound of Formula A: ND, compound of Formula B: ND, compound of Formula C: ND, compound of Formula D: ND, compound of Formula E: ND, compound of Formula F: ND.

### Example 5(E)

To a 4 neck round bottom flask, trofinetide (15 gm) was dissolved in methanol (45 mL). Above solution was charged slowly into 2-methyltetrahydrofuran (150 mL) at ambient temperature and stirred for 30 min. Reaction mass was filtered and washed with 2-methyltetrahydrofuran (75 mL) and dried under vacuum to obtain trofinetide. Yield: 10.2 g (85%), Purity: 99.74%, compound of Formula A: ND, compound of Formula B: ND, compound of Formula C: ND, compound of Formula D: ND, compound of Formula E: ND, compound of Formula F: ND.

### Example 5(F)

To a 4 neck round bottom flask, trofinetide (100 gm) was dissolved in methanol (300 mL). Above solution was charged slowly into 2-methyltetrahydrofuran (1000 mL) at ambient temperature and stirred for 30 min. Reaction mass was filtered and washed with 2-methyltetrahydrofuran (500 mL) and dried under vacuum to obtain trofinetide. Yield: 68 g (85%), Purity: 99.70%, compound of Formula A: ND, compound of Formula B: ND, compound of Formula C: ND, compound of Formula D: ND, compound of Formula E: 0.11%, compound of Formula F: 0.12%.

### Example 5(G)

To a 4 neck round bottom flask, trofinetide (5 gm) was dissolved in methanol (10 mL). Above solution was slowly charged into M TBE (50 mL) at ambient temperature and stirred for 1 hour. Reaction mass was filtered, washed with MTBE (25 mL) and dried under vacuum to obtain trofinetide. Yield: 4.8 g (96%), Purity: 98.67%, compound of Formula A: ND, compound of Formula B: ND, compound of Formula C: 0.10%, compound of Formula D: 0.11%, compound of Formula E: 0.12%, compound of Formula F: ND.

### Example 5(H)

To a 4 neck round bottom flask, trofinetide (5 gm) was dissolved in methanol (10 mL). Above solution was slowly charged into diethyl ether (35 mL) at ambient temperature and stirred for 1 hour. Reaction mass was filtered, washed with diethyl ether (12.5 mL) and dried under vacuum to obtain trofinetide. Yield: 4.85 g (97%), Purity: 98.56, compound of Formula A: ND, compound of Formula B: ND, compound of Formula C: 0.24%, compound of Formula D: ND, compound of Formula E: 0.24%, compound of Formula F: ND.

While the present invention has been described in terms of its specific embodiments, certain modification and equivalents will be apparent to those skilled in art and the intended to be included within the scope of the invention.

1-11. (canceled)

12. Trofinetide having a purity of 99.0% or more and one or more of each of compound of Formula II, compound of Formula A, compound of Formula B, compound of Formula C, compound of Formula D, compound of Formula E, or compound of Formula F, 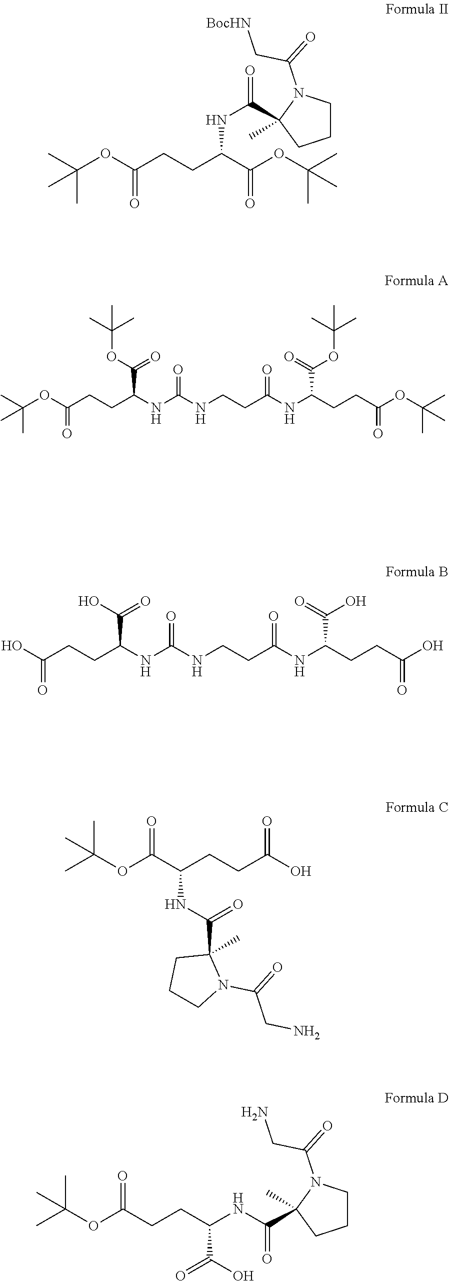 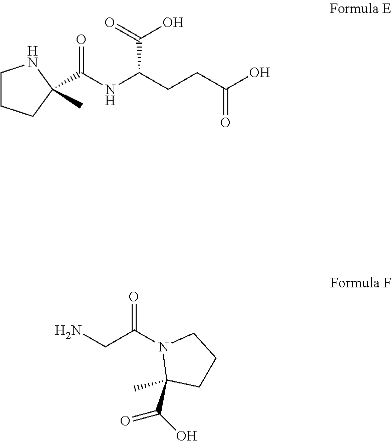 [SMILES: C[C@@]1(C(=O)N[C@@H](CCC(=O)O)C(=O)O)CCCN1 ; C[C@@]1(C(=O)O)CCCN1C(=O)CN]
which when present, is in a detectable amount of about 0.15% or less, as measured by area percentage of HPLC.

13. The trofinetide according to claim 12, wherein the trofinetide is crystalline or amorphous.

14. The trofinetide according to claim 12, wherein the trofinetide is in the form of salts, hydrates, or solvates thereof.

15. A composition comprising trofinetide having a purity of 99.0% or more, as measured by area percentage of HPLC.

16. The composition according to claim 15, wherein the trofinetide is having a purity of 99.0% or more and one or more of each of compound of Formula II, compound of Formula A, compound of Formula B, compound of Formula C, compound of Formula D, compound of Formula E or compound of Formula F, 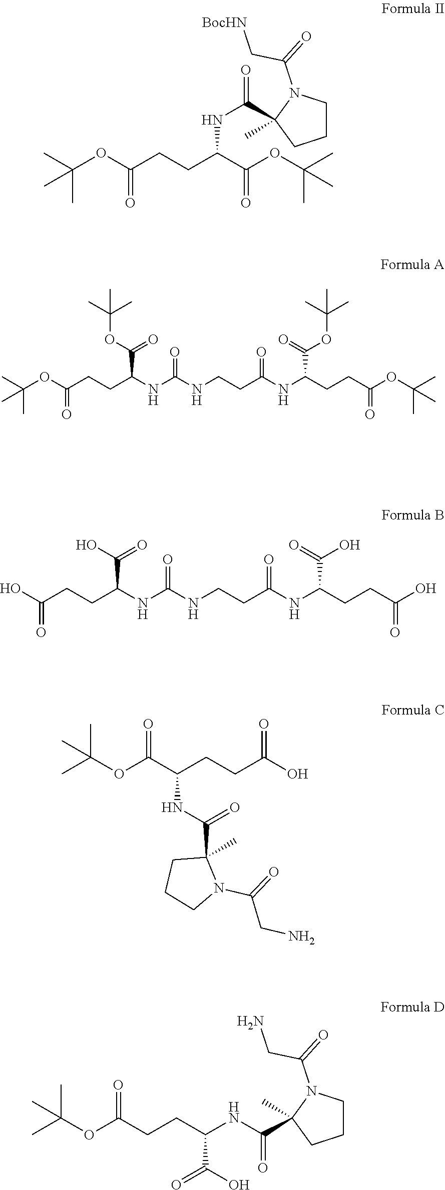 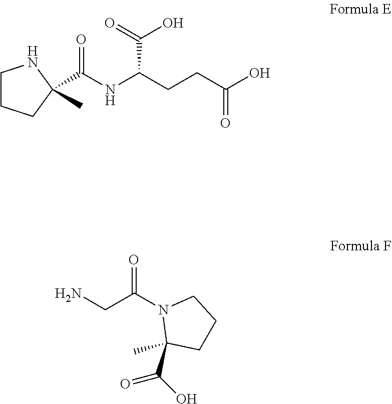 [SMILES: C[C@@]1(C(=O)N[C@@H](CCC(=O)O)C(=O)O)CCCN1 ; C[C@@]1(C(=O)O)CCCN1C(=O)CN]
which when present, is in a detectable amount of about 0.15% or less, as measured by area percentage of HPLC.

17. The composition according to claim 16 is a pharmaceutical composition together with pharmaceutically acceptable excipients, diluents, and carriers.

18. The pharmaceutical composition according to claim 17, wherein the trofinetide is in the form of salts, hydrates, or solvates thereof.

19. A process for the preparation of trofinetide, or salts, or hydrates thereof, the process comprising:
(a) reacting a compound of Formula VII, 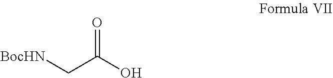 [SMILES: CC(C)(C)OC(=O)NCC(=O)O]
with N-Hydroxysuccinimide in the presence of one or more reagents to obtain a compound of Formula VI; 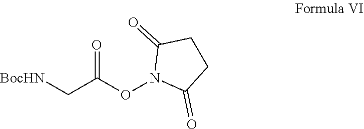 [SMILES: CC(C)(C)OC(=O)NCC(=O)ON1C(=O)CCC1=O]
(b) reacting the compound of Formula VI with a compound of Formula V or salts thereof, 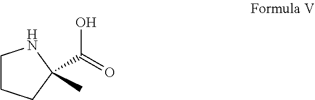
in the presence of one or more bases to obtain a compound of Formula IV; 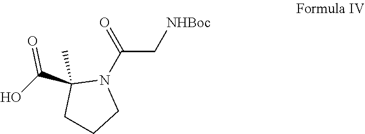 [SMILES: CC(C)(C)OC(=O)NCC(=O)N1CCC[C@@]1(C)C(=O)O]
(c) reacting the compound of Formula IV with a compound of Formula III or salts thereof, 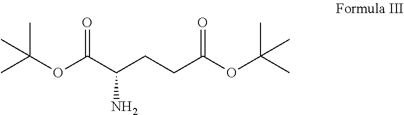 [SMILES: CC(C)(C)OC(=O)CC[C@H](N)C(=O)OC(C)(C)C]
in the presence of one or more reagents to obtain a compound of Formula II; 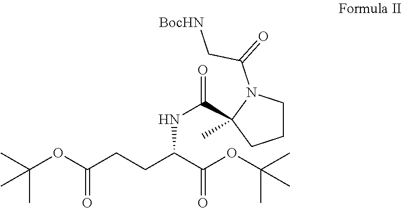 [SMILES: CC(C)(C)OC(=O)CC[C@H](NC(=O)[C@]1(C)CCCN1C(=O)CNC(=O)OC(C)(C)C)C(=O)OC(C)(C)C]
(d) deprotecting the compound of Formula II with a reagent to obtain trofinetide and
(e) optionally, converting trofinetide obtained in step (d) to salts or hydrates thereof.

20. The process according to claim 19, wherein the reagent at step (a) is selected from one or more of 1-hydroxy-benzotriazole (HOBt), 1-hydroxy-azabenzotriazole (HOAt), hexafluorophosphate azabenzotriazole tetramethyl uronium (HATU), hexafluorophosphate benzotriazole tetramethyl uronium (HBTU), 2-(1H-benzotriazole-1-yl)-1,1,3,3-tetramethylaminium tetrafluoroborate (TBTU), O-(1,2-dihydro-2-oxo-1-pyridyl)-N,N,N′-N′-tetramethyluronium tetrafluoroborate (TPTU), O-((ethoxycarbonyl)cyanomethyleneamino)-N,N,N′,N′-tetramethyluronium tetrafluoroborate (TOTU), 1-Ethyl-3-(3-dimethylaminopropyl)carbodiimide (EDC), 1-ethyl-3-(3-dimethylaminopropyl)carbodiimide hydrochloride (EDC.HCl), N,N′-dicyclohexylcarbodiimide (DCC), N,N′-diisopropylcarbodiimide (DIC), or mixtures thereof.

21. The process according to claim 19, wherein the reactions at step (a), (b), (c), (d) and (e) may be performed in the presence of one or more solvents selected from one or more of acetonitrile, tetrahydrofuran, 2-methyltetrahydrofuran, xylene, methanol, ethanol, propanol, isopropyl alcohol, dichloromethane, water, toluene, ethyl acetate, dimethylformamide, dimethylacetamide, N-M ethyl-2-pyrrolidone, n-heptane, n-hexane, dioxane, di-n-butyl ether, methyl tert-butyl ether, dimethyl ether, or mixtures thereof.

22. The process according to claim 19, wherein the base at step (b) is selected from one or more of triethyl amine (TEA), N,N-diisopropylethylamine (DIPEA), N-methylmorpholine (NMM), pyridine, N-methylpiperidine, or mixtures thereof.

23. The process according to claim 19, wherein the reagent at step (c) is selected from one or more of HOBt, HOAt, HATU, HBTU, TBTU, TPTU, TOTU, EDC, EDC.HCl, DCC, DIC, 4-dimethylaminopyridine (DMAP), TEA, DIPEA, NMM, pyridine, N-methylpiperidine, or mixtures thereof.

24. The process according to claim 19, wherein the reagent at step (d) is selected from one or more of trifluoroacetic acid, hydrochloric acid, trimethylsilyl iodide (TMSI), or mixtures thereof.

25. A compound of Formula II,

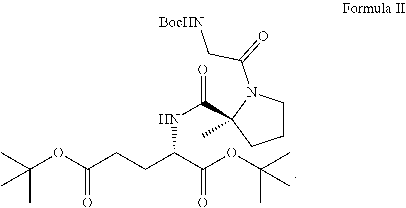 [SMILES: CC(C)(C)OC(=O)CC[C@H](NC(=O)[C@]1(C)CCCN1C(=O)CNC(=O)OC(C)(C)C)C(=O)OC(C)(C)C]

26. A compound of Formula II according to claim 25, 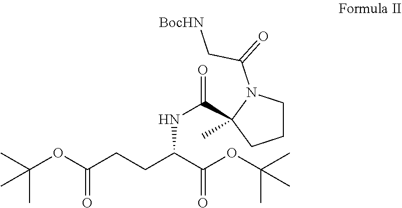 [SMILES: CC(C)(C)OC(=O)CC[C@H](NC(=O)[C@]1(C)CCCN1C(=O)CNC(=O)OC(C)(C)C)C(=O)OC(C)(C)C]
used in the preparation of trofinetide or salts, or hydrates thereof.
